(19) United States
(12) Patent Application Publication
(10) Pub. No.: US 2009/0082394 A1
(43) Pub. Date: Mar. 26, 2009

(54) Tetrahydroisoquinoline Derivatives to Enhance Memory Function

(75) Inventor: Francois Jenck, Riedisheim (FR)

Correspondence Address: HOXIE & ASSOCIATES LLC, 75 MAIN STREET , SUITE 301, MILLBURN, NJ, 07041, US

(73) Assignee: ACTELION PHARMACEUTICALS, LTD., Allschwil (CH)

(21) Appl. No.: 12/293,031

(22) PCT Filed: Mar. 14, 2007
(86) PCT No.: PCT/IB2007/050868

§ 371 Date: Sep. 15, 2008

(30) Foreign Application Priority Data
Mar. 15, 2006 (IB) PCT/IB2006/050812

(51) Int. Cl. A61K 31/472; A61P 25/28

(52) U.S. Cl. 514/307

(57) ABSTRACT

The invention relates to the use of tetrahydroisoquinoline derivatives for the preparation of a medicament to enhance, maintain and/or restore all stages and/or types of short-, middle- and/or long-term memory.

The present invention provides methods for enhancing short-, middle- and/or long-term memory function and performance, either preventively or curatively, to enhance basal levels, prevent deficits or to restore capabilities in learning and memory deficits.

The present invention provides also methods for enhancing short-, middle- and long-term memory function and performance, either preventively or curatively, to enhance basal learning & memory function, to slow down and prevent deficits or to restore capabilities in learning and memory deficits.

Specifically, the present invention provides known tetrahydroisoquinoline derivatives of the general formula I for enhancing short-, middle- and/or long-term memory function and performance, either preventively or curatively to enhance basal levels, prevent deficits or to restore capabilities in learning and memory deficits. Additionally, the present invention provides known tetrahydroisoquinoline derivatives of the general formula I for enhancing short-, middle- and/or long-term memory function and performance, either preventively or curatively, to enhance basal learning & memory function, to slow down and prevent deficits or to restore capabilities in learning and memory deficits.

Orexin receptor antagonists (collectively referred to herein as “OXRA compounds”) are a novel type of nervous system or psychotropic drugs that decrease alertness and promote sleep. Their mode of action in animals and humans involves blockade of orexin receptors in the brain and modulation of sleep and arousal systems. OXRAs are currently developed for use in the treatment of sleep disorders and insomnias.

Human memory is a set of complex and interrelated forms of reminiscences most commonly divided into declarative forms, with further subdivisions into episodic and semantic memory; and non-declarative forms, subdivided into an array of different types including procedural skill memory. Declarative recall is, e.g., for facts and events accessible to conscious recollection, and non-declarative recall is, e.g. procedural memory of skills and operations. A newly acquired experience initially is susceptible to various forms of disruption. With time, however, the new experience becomes resistant to disruption. This observation has been interpreted to indicate that a labile, working, short-term memory is consolidated into a more stable, long-term memory. Following the initial encoding of a memory, several ensuing stages are proposed: consolidation, integration of the memory representation, translocation of the representation, or erasure of the memory. Following later recall, the memory representation is believed to become unstable once again, requiring periods of reconsolidation. Behavioural research has found that the human mind consolidates memory at certain key time intervals. The initial phase of memory consolidation occurs in the first few minutes after we are exposed to a new idea or learning experience. The next phase occurs over a longer period of time, such as during sleep. If a learning experience has ongoing meaning to us, the next week or so serves as a further period of memory consolidation. In effect, in this phase, the memory moves from short-term to long-term storage, or the memory moves from short-term to middle-term and from middle-term to long-term storage.

Memory consolidation, or long-term memory is believed to be fundamentally affected in a variety of neurological and mental disorders, such as e.g. mental retardation, Alzheimer's disease or depression. Indeed, loss or impairment of long-term memory is a significant feature of such diseases, and no effective therapy to prevent long-term memory loss has emerged yet. Short-term, or “working” memory is generally not significantly impaired in such patients.

It is speculated in the prior art, that orexin receptor antagonists may improve the memory capacity (Presentation by Actelion Jan. 11, 2006, as well as articles published in February 2006). At that time no information was given on the structural class of compounds that may demonstrate activity to improve memory capacity, nor on what stage and type of memory process could be involved.

The present invention relates to the discovery that the orexin receptor antagonist of the general formula I may affect beneficially all or any of these forms and stages of memory.

These compounds are of potential use to enhance and/or restore short-, middle- and/or long-term memory function and performance. In particular, these compounds are of potential use to enhance and/or restore long-term memory function and performance, e.g., to improve long-term memory.

It is an object of the present invention to provide methods and composition for enhancing long-term memory function and performance, either preventively or curatively to enhance basal levels, prevent deficits or to restore capabilities in learning and memory deficits. It is another object of the present invention to provide methods and composition for enhancing long-term memory function and performance, either preventively or curatively, to enhance basal learning & memory function, to slow down and prevent deficits or to restore capabilities in learning and memory deficits.

The present invention relates to a method for enhancing general memory, comprising administering a formulation of an orexin receptor antagonist of the general formula (I), or a pharmaceutically acceptable derivative, salt, solvate, prodrug or metabolic derivative thereof, in an amount sufficient to enhance memory.

The present invention relates to the discovery that the orexin receptor antagonist of the general formula (I) may affect all or any of these forms and stages of memory. In particular, these compounds are of potential use to enhance and/or restore long-term memory function and performance, e.g., to improve long-term memory.

The synthesis of the tetrahydroisoquinoline derivatives of the general formula (I) is described in WO 01/68609, WO 2004/085403 and WO 2005/118548.

The present invention relates to the use of compounds of general formula (I) for the preparation of a medicament to enhance, maintain and/or restore all stages and/or types of short-, middle- and/or long-term memory,

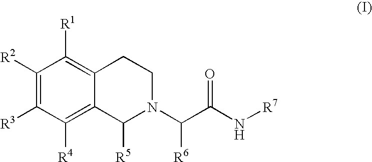

wherein
R¹, R², R³, R⁴ independently represent cyano, halogen, hydrogen, hydroxy, (C₁₋₄)alkyl, (C₂₋₄)alkenyl, (C₁₋₄)alkoxy, (C₂₋₄)alkenyloxy, trifluoromethyl, trifluoromethoxy, (C₃₋₆)cycloalkyloxy, aryloxy, aryl-(C₁₋₄)alkoxy, heteroaryloxy, heteroaryl-(C₁₋₄)alkoxy, R⁸CO—, NR⁹R¹⁰CO—, NR⁹R¹⁰COO—, R⁹R¹⁰N—, R⁸OOC—, R⁸SO₂NH— or R¹¹CO—NH— or R¹ and R² together or R² and R³ together or R³ and R⁴ together may form with the phenyl ring, to which they are attached, a five, six or seven-membered ring containing one or two oxygen atoms;
R⁵ represents hydrogen, aryl, aryl-(C₁₋₄)alkyl, aryl-(C₂₋₄)alkenyl, aryl-oxy-(C₁₋₄)alkyl, heteroaryl-(C₁₋₄)alkyl or heteroaryl-oxy-(C₁₋₄)alkyl;
R⁶ represents hydrogen, aryl or heteroaryl;
R⁷ represents hydrogen, (C₁₋₄)alkyl, (C₂₋₄)alkenyl, (C₃₋₆)cycloalkyl, (C₃₋₆)cycloalkyl-(C₁₋₄)alkyl, aryl, aryl-(C₁₋₄)alkyl, or heteroaryl-(C₁₋₄)alkyl; or R⁷ represents an indanyl-, a 1,2,3,4-tetrahydro-naphthalenyl, or a 6,7,8,9-tetrahydro-5H-benzocycloheptenyl-group which groups might be unsubstituted, or substituted in the saturated ring with (C₁₋₄)alkyl, hydroxy, or phenyl, or substituted in the aromatic ring with one, two or three substituents independently selected from (C₁₋₄)alkyl, (C₁₋₄)alkoxy or halogen;
R⁸ represents (C₁₋₄)alkyl, aryl, aryl-(C₁₋₄)alkyl, heteroaryl or heteroaryl-(C₁₋₄)alkyl;
R⁹ and R¹⁰ independently represent hydrogen, (C₁₋₄)alkyl, (C₃₋₆)cycloalkyl, aryl, aryl-(C₁₋₄)alkyl, heteroaryl or heteroaryl-(C₁₋₄)alkyl or R⁹ and R¹⁰ together with the nitrogen atom, to which they are attached, may form a five or six-membered saturated ring such as a pyrrolidine or a piperidine ring;
R¹¹ represents (C₁₋₄)alkyl, aryl, (C₃₋₆)cycloalkyl, heteroaryl, R⁹R¹⁰N— or R⁸O—.

A further embodiment of the invention is the use of compounds of the general formula (I) as defined above, wherein

R¹ and R⁴ represent hydrogen;
R² and R³ independently represent hydrogen, hydroxy, (C₁₋₄)alkyl, (C₁₋₄)alkoxy, trifluoromethoxy, (C₃₋₆)cycloalkyloxy, aryl-(C₁₋₄)alkoxy, heteroaryloxy or NR⁹R¹⁰COO—;
R⁵ represents aryl-(C₁₋₁₄)alkyl or heteroaryl-(C₁₋₄)alkyl;
R⁶ represents hydrogen, aryl or heteroaryl;
R⁷ represents hydrogen, (C₁₋₄)alkyl, (C₂₋₄)alkenyl, (C₃₋₆)cycloalkyl, (C₃₋₆)cycloalkyl-(C₁₋₄)alkyl, aryl-(C₁₋₄)alkyl or heteroaryl-(C₁₋₄)alkyl; or R⁷ represents an indanyl-, a 1,2,3,4-tetrahydro-naphthalenyl, or a 6,7,8,9-tetrahydro-5H-benzocycloheptenyl-group which groups might be unsubstituted, or substituted in the saturated ring with (C₁₋₄)alkyl, hydroxy, or phenyl, or substituted in the aromatic ring with one, two or three substituents independently selected from (C₁₋₄)alkyl, (C₁₋₄)alkoxy or halogen;
R⁹ and R¹⁰ independently represent hydrogen or (C₁₋₄)alkyl or R⁹ and R¹⁰ together with the nitrogen atom, to which they are attached, may form a five or six-membered saturated ring.

A further embodiment of the invention is the use of compounds of the general formula (I) as defined above, wherein

R¹ and R⁴ represent hydrogen;
R² and R³ independently represent hydrogen, (C₁₋₄)alkoxy or (C₃₋₆)cycloalkyloxy;
R⁵ represents aryl-(C₁₋₄)alkyl or heteroaryl-(C₁₋₄)alkyl;
R⁶ represents aryl or heteroaryl;
R⁷ represents hydrogen, (C₁₋₄)alkyl, (C₃₋₆)cycloalkyl or (C₃₋₆)cycloalkyl-(C₁₋₄)alkyl.

A further embodiment of the invention is the use of compounds of the general formula (I) as defined above, wherein

R¹ and R⁴ represent hydrogen;
R² and R³ independently represent (C₁₋₄)alkoxy;
R⁵ represents aryl-(C₁₋₄)alkyl or heteroaryl-(C₁₋₄)alkyl;
R⁶ represents a phenyl group;
R⁷ represents hydrogen or (C₁₋₄)alkyl.

A further embodiment of the invention is the use of compounds of the general formula (I) as defined above, wherein

R¹ and R⁴ represent hydrogen;
R² and R³ represent methoxy;
R⁵ represents a 2-phenyl-ethyl- or a 2-pyridyl-ethyl group which groups are substituted with one or two substituents independently selected from methyl, trifluoromethyl or halogen;
R⁶ represents a phenyl group;
R⁷ represents hydrogen or (C₁₋₄)alkyl.

The above-mentioned compounds of general formula (I) are also useful for the preparation of a medicament to enhance and/or restore long-term memory function and performance.

In the present description the term (C₁₋₄)alkyl, alone or in combination, means a straight-chain or branched-chain alkyl group with 1 to 4 carbon atoms which might be unsubstituted or substituted with cyano, a (C₁₋₄)alkoxycarbonyl group or one, two or three fluorine atoms. Examples of straight-chain and branched (C₁₋₄)alkyl groups are methyl, ethyl, n-propyl, isopropyl, n-butyl, isobutyl, sec-butyl, tert-butyl, cyanomethyl and 2-cyanoethyl. Preferred (C₁₋₄)alkyl groups are methyl, n-butyl and sec.-butyl. Especially preferred (C₁₋₄)alkyl group is methyl.

The term (C₂₋₄)alkenyl, alone or in combination, means a straight-chain or branched-chain alkenyl group with 2 to 4 carbon atoms, preferably allyl and vinyl.

The term (C₁₋₄)alkoxy, alone or in combination, means a group of the formula (C₁₋₄)alkyl-O— in which the term (C₁₋₄)alkyl has the previously given significance, such as methoxy, ethoxy, n-propoxy, isopropoxy, n-butoxy, isobutoxy, sec-butoxy and tert-butoxy. The (C₁₋₄)alkyl group might be unsubstituted or substituted with a (C₃₋₆)cycloalkyl group, a (C₁₋₄)alkoxycarbonyl group or one, two or three fluorine atoms. Examples of substituted (C₁₋₄)alkoxy groups are cyclopropylmethoxy, 2-fluoro-ethoxy, 2,2-difluoro-ethoxy and 3-fluoro-propoxy. Preferred substituted or unsubstituted (C₁₋₄)alkoxy groups are methoxy, ethoxy, n-propoxy, isopropoxy, tert.-butoxy, cyclopropylmethoxy, 2-fluoro-ethoxy, 2,2-difluoro-ethoxy and 3-fluoro-propoxy. Especially preferred is methoxy.

For the substituent R¹, the term “(C₁₋₄)alkoxy” means preferably methoxy and n-propoxy.

For the substituent R², the term “(C₁₋₄)alkoxy” means preferably methoxy.

For the substituent R³, the term “(C₁₋₄)alkoxy” means preferably methoxy, ethoxy, n-propoxy, isopropoxy, tert.-butoxy, cyclopropylmethoxy, 2-fluoro-ethoxy, 2,2-difluoro-ethoxy and 3-fluoro-propoxy. More preferred are methoxy, ethoxy, isopropoxy and 2,2-difluoro-ethoxy.

For the substituent R⁴, the term “(C₁₋₄)alkoxy” means preferably methoxy, ethoxy, n-propoxy, isopropoxy, 2-fluoro-ethoxy and 2,2-difluoro-ethoxy.

The term (C₁₋₄)alkoxycarbonyl, alone or in combination, means a group (C₁₋₄)alkoxy-(CO)—, wherein the term (C₁₋₄)alkoxy has the previously given significance. Examples are methoxycarbonyl or ethoxycarbonyl.

The term (C₂₋₄)alkenyloxy, alone or in combination, means a group of the formula (C₂₋₄)alkenyl-O— in which the term (C₂₋₄)alkenyl has the previously given significance, such as vinyloxy and allyloxy.

The term (C₃₋₆)cycloalkyl, alone or in combination, means a cycloalkyl ring with 3 to 6 carbon atoms. Examples of (C₃₋₆)cycloalkyl are cyclopropyl, cyclobutyl, cyclopentyl and cyclohexyl, preferably cyclopropyl. The (C₃₋₆)cycloalkyl group might be unsubstituted or substituted with one or two methyl groups. Examples are methyl-cyclopropyl, dimethyl-cyclopropyl, methyl-cyclobutyl, methyl-cyclopentyl, methyl-cyclohexyl or dimethyl-cyclohexyl.

The term (C₃₋₆)cycloalkyloxy, alone or in combination, means a group of the formula (C₃₋₆)cycloalkyl-O— in which the term (C₃₋₆)cycloalkyl has the previously given significance, such as cyclopropyloxy, cyclobutyloxy, cyclopentyloxy or cyclohexyloxy. Preferred are cyclopropyloxy and cyclohexyloxy.

The term (C₃₋₆)cycloalkyl-(C₁₋₄)alkyl, alone or in combination, means a (C₁₋₄)alkyl group as previously defined in which one hydrogen atom has been replaced by an (C₃₋₆)cycloalkyl group as previously defined. Examples of (C₃₋₆)cycloalkyl-(C₁₋₄)alkyl groups are cyclopropyl-methyl and cyclohexyl-methyl.

The term (C₃₋₆)cycloalkyl-(C₁₋₄)alkoxy, alone or in combination, means a (C₁₋₄)alkoxy group as previously defined in which one hydrogen atom has been replaced by an (C₃₋₆)cycloalkyl group as previously defined. Examples of (C₃₋₆)cycloalkyl-(C₁₋₄)alkoxy groups are cyclopropyl-methoxy and cyclohexyl-methoxy. Preferred is cyclopropyl-methoxy.

The term aryl, alone or in combination, means a phenyl or naphthyl group which optionally carries one, two or three substituents, each independently selected from cyano, halogen, hydroxy, (C₁₋₄)alkyl, (C₂₋₄)alkenyl, (C₁₋₄)alkoxy, (C₂₋₄)alkenyloxy, (C₃₋₆)cycloalkyl-(C₁₋₁₄)alkoxy, heteroaryloxy, trifluoromethyl, difluoromethoxy, trifluoromethoxy, amino, NR⁹R¹⁰COO— and R⁹R¹⁰N—. Preferred substituents are halogen, (C₁₋₄)alkyl, (C₁₋₄)alkoxy and trifluoromethyl. Additionally the aryl ring, if equal to phenyl, may be part of a benzo[1,3]dioxole group. Examples of aryl groups are 2-fluoro-phenyl, 3-fluoro-phenyl, 4-fluoro-phenyl, 2-chloro-phenyl, 3-chloro-phenyl, 4-chloro-phenyl, 2-methyl-phenyl, 3-methyl-phenyl, 4-methyl-phenyl, 2-methoxy-phenyl, 3-methoxy-phenyl and 4-methoxy-phenyl. Preferred aryl group is phenyl.

The term aryloxy, alone or in combination, means a group of the formula aryl-O— in which the term aryl has the previously given meaning. Examples of aryloxy groups are phenoxy, 3-trifluoromethyl-phenoxy and 4-trifluoromethyl-phenoxy. Preferred is phenoxy.

The term aryl-oxy-(C₁₋₄)alkyl, alone or in combination, means a group of the formula (C₁₋₄)alkyl attached to the oxygen atom of aryl-O—. The terms aryl and (C₁₋₄)alkyl have the previously given meaning. Examples of aryl-oxy-(C₁₋₄)alkyl groups are phenoxy-methyl, 3-trifluoromethyl-phenoxy-methyl and 4-trifluoromethyl-phenoxy-methyl. Preferred is phenoxy-methyl.

The term aryl-(C₁₋₄)alkyl, alone or in combination, means an (C₁₋₄)alkyl group as previously defined in which one hydrogen atom has been replaced by an aryl group as previously defined. Examples of aryl-(C₁₋₄)alkyl groups are benzyl, naphth-1-ylmethyl, naphth-2-ylmethyl, 2-(naphth-1-yl)-ethyl and 2-phenyl-ethyl which groups might be unsubstituted or substituted in the aryl group with one, two or three substituents independently selected from methoxy, difluoromethoxy, trifluoromethoxy, ethoxy, propoxy, 3-fluoro-propoxy, isopropoxy, iso-butoxy, cyclopropylmethoxy, allyloxy, benzyloxy, methyl, trifluoromethyl, ethyl, tert.-butyl, fluorine, chlorine, bromine, dimethylamino and hydroxy.

For the substituent R⁵, the term “aryl-(C₁₋₄)alkyl” means preferably 3,4-dimethoxy-benzyl, 3-ethoxy-4-methoxy-benzyl, 4-cyclopropylmethoxy-3-methoxy-benzyl, 3-methoxy-4-(2-methyl-propoxy)-benzyl, 3-fluoro-4-methoxy-benzyl, 3,4-dimethyl-benzyl, 3,4-diethyl-benzyl, 3,4-dichloro-benzyl, 2-(2-fluoro-phenyl)-ethyl, 2-(2,3,4-trifluoro-phenyl)-ethyl, 2-(2,3,5-trifluoro-phenyl)-ethyl, 2-(2,3,6-trifluoro-phenyl)-ethyl, 2-(3-chloro-2-fluoro-phenyl)-ethyl, 2-(3-methyl-phenyl)-ethyl, 2-(4-methyl-phenyl)-ethyl, 2-(3,4-dimethyl-phenyl)-ethyl, 2-(2-fluoro-3-methyl-phenyl)-ethyl, 2-(3-fluoro-4-methyl-phenyl)-ethyl, 2-(4-fluoro-3-methyl-phenyl)-ethyl, 2-(3-chloro-4-methyl-phenyl)-ethyl, 2-(2-difluoromethoxy-phenyl)-ethyl, 2-(2-trifluoromethoxy-phenyl)-ethyl, 2-(3-trifluoromethoxy-phenyl)-ethyl, 2-(3-trifluoromethyl-phenyl)-ethyl, 2-(4-trifluoromethyl-phenyl)-ethyl, 2-(2-fluoro-3-trifluoromethyl-phenyl)-ethyl, 2-(2-fluoro-4-trifluoromethyl-phenyl)-ethyl and 2-(3-fluoro-4-trifluoromethyl-phenyl)-ethyl. More preferred are 3,4-dimethoxy-benzyl, 3,4-dimethyl-benzyl, 3,4-diethyl-benzyl, 2-(2,3,6-trifluoro-phenyl)-ethyl, 2-(4-methyl-phenyl)-ethyl, 2-(2-fluoro-3-methyl-phenyl)-ethyl, 2-(3-fluoro-4-methyl-phenyl)-ethyl, 2-(4-trifluoromethyl-phenyl)-ethyl, 2-(2-fluoro-4-trifluoromethyl-phenyl)-ethyl and 2-(3-fluoro-4-trifluoromethyl-phenyl)-ethyl. Particularly preferred is 2-(4-trifluoromethyl-phenyl)-ethyl. For the substituent R⁷, the term “aryl-(C₁₋₄)alkyl” means preferably benzyl, naphth-1-yl-methyl, 2-methylbenzyl, 2-methoxybenzyl, 2-ethoxybenzyl and benzo[1,3]dioxol-5-yl-methyl. More preferred are benzyl, naphth-1-yl-methyl and benzo[1,3]dioxol-5-yl-methyl.

The term aryl-(C₁₋₄)alkoxy, alone or in combination, means a (C₁₋₄)alkoxy group as previously defined in which one hydrogen atom has been replaced by an aryl group as previously defined. Examples of aryl-(C₁₋₄)alkoxy groups are benzyloxy, naphth-1-yl-methoxy and naphth-2-yl-methoxy. Preferred is benzyloxy.

The term aryl-(C₂₋₄)alkenyl, alone or in combination, means an (C₂₋₄)alkenyl group as previously defined in which one hydrogen atom has been replaced by an aryl group as previously defined. Examples of aryl-(C₂₋₄)alkenyl groups are 2-phenyl-ethenyl and 2-naphthyl-ethenyl which groups might be unsubstituted or substituted in the aryl group with one, two or three substituents independently selected from (C₁₋₄)alkyl, (C₁₋₄)alkoxy, trifluoromethyl and halogen. Preferred are 2-phenyl-ethenyl groups, which groups might be unsubstituted or substituted in the aryl group with one or two substituents independently selected from methyl, methoxy, trifluoromethyl, fluorine and chlorine. More preferred are 2-(2,3-difluorophenyl)-ethenyl and 2-(2,5-difluorophenyl)-ethenyl.

The term heteroaryl, alone or in combination, means a 5- to 10-membered monocyclic or bicyclic aromatic ring containing 1, 2 or 3 heteroatoms selected from oxygen, nitrogen and sulphur which may be the same or different. The heteroaryl group might be unsubstituted or substituted with up to three substituents independently selected from cyano, halogen, hydroxy, (C₁₋₄)alkyl, (C₂₋₄)alkenyl, (C₁₋₄)alkoxy, (C₂₋₄)alkenyloxy, trifluoromethyl, trifluoromethoxy, or amino. Examples of such heteroaryl groups are pyridyl, pyrimidinyl, pyrazinyl, pyridazinyl, quinolinyl, isoquinolinyl, thienyl, thiazolyl, isothiazolyl, furyl, imidazolyl, pyrazolyl, pyrrolyl, indazolyl, indolyl, isoindolyl, benzimidazolyl, isoxazolyl, oxazolyl, oxadiazolyl, thiadiazolyl, quinoxalinyl, phthalazinyl, cinnolinyl, isobenzofuranyl. A preferred heteroaryl group is pyridyl, which might be unsubstituted or substituted with methyl, ethyl or methoxy.

The term heteroaryloxy, alone or in combination, means a group of the formula heteroaryl-O— in which the term heteroaryl has the previously given meaning. Examples of heteroaryloxy groups are pyridin-2-yloxy, pyrimidin-2-yloxy, pyrazin-2-yloxy and thiazol-2-yloxy which groups might be unsubstituted or substituted with one or two substituents independently selected from (C₁₋₄)alkyl, (C₁₋₄)alkoxy, trifluoromethyl and halogen. Preferred are 5-chloro-pyridin-2-yloxy, pyrimidin-2-yloxy, 5-methyl-pyrimidin-2-yloxy, 4,6-dimethyl-pyrimidin-2-yloxy, 5-methoxy-pyrimidin-2-yloxy, 5-bromo-pyrimidin-2-yloxy, 4-trifluoromethyl-pyrimidin-2-yloxy, pyrazin-2-yloxy and thiazol-2-yloxy.

The term heteroaryl-oxy-(C₁₋₄)alkyl, alone or in combination, means a group of the formula (C₁₋₄)alkyl attached to the oxygen atom of heteroaryl-O—. The terms heteroaryl and (C₁₋₄)alkyl have the previously given meaning. Examples of heteroaryl-oxy-(C₁₋₄)alkyl groups are (pyridin-2-yloxy)-methyl and (pyridin-3-yloxy)-methyl which groups might be unsubstituted or substituted with one or two substituents independently selected from (C₁₋₄)alkyl, (C₁₋₄)alkoxy, trifluoromethyl and halogen. Preferred is (6-trifluoromethyl-pyridin-3-yloxy)-methyl.

The term heteroaryl-(C₁₋₄)alkyl, alone or in combination, means an (C₁₋₄)alkyl group as previously defined in which one hydrogen atom has been replaced by an heteroaryl group as previously defined. Examples of heteroaryl-(C₁₋₄)alkyl groups are pyridin-2-ylmethyl, pyridin-3-ylmethyl, pyridin-4-ylmethyl, furan-2-ylmethyl, benzimidazol-2-ylmethyl, 2-(pyridin-2-yl)-ethyl, 2-(pyridin-3-yl)-ethyl and 2-(furan-3-yl)-ethyl which groups might be unsubstituted or substituted with one or two substituents independently selected from (C₁₋₄)alkyl, (C₁₋₄)alkoxy, trifluoromethyl and halogen.

For the substituent R⁵, the term “heteroaryl-(C₁₋₄)alkyl” means preferably 2-(pyridin-3-yl)-ethyl substituted with methyl, methoxy, chlorine and trifluoromethyl. Particularly preferred is 2-(6-trifluoromethyl-pyridin-3-yl)-ethyl.

For the substituent R⁷, the term “heteroaryl-(C₁₋₄)alkyl” means preferably pyridin-2-ylmethyl, pyridin-3-ylmethyl, pyridin-4-ylmethyl, furan-2-ylmethyl and benzimidazol-2-ylmethyl. Particularly preferred is pyridin-2-ylmethyl.

The term heteroaryl-(C₁₋₄)alkoxy, alone or in combination, means an (C₁₋₄)alkoxy group as previously defined in which one hydrogen atom has been replaced by an heteroaryl group as previously defined as for example pyridyl-methoxy.

The term halogen means fluorine, chlorine, bromine or iodine and preferably fluorine and chlorine.

The term “indanyl” means an indanyl group which might be unsubstituted, or substituted in the saturated ring with (C₁₋₄)alkyl, hydroxy, or phenyl, or substituted in the aromatic ring with one, two or three substituents independently selected from (C₁₋₄)alkyl, (C₁₋₄)alkoxy or halogen. Examples of indanyl groups are indan-1-yl, indan-2-yl, 2-hydroxy-indan-1-yl, 2-methyl-indan-1-yl, 3-methyl-indan-1-yl, 3-phenyl-indan-1-yl, 4-methyl-indan-1-yl, 4-methoxy-indan-1-yl, 5-methoxy-indan-1-yl, 5,6-dimethoxy-indan-1-yl, 5-fluoro-indan-1-yl, 5-bromo-indan-1-yl, 6-methyl-indan-1-yl and 6-methoxy-indan-1-yl. Preferred are indan-1-yl, 4-methyl-indan-1-yl, 4-methoxy-indan-1-yl, 5-methoxy-indan-1-yl, 6-methyl-indan-1-yl and 6-methoxy-indan-1-yl. Particularly preferred is indan-1-yl.

The term “1,2,3,4-tetrahydro-naphthalenyl” means a 1,2,3,4-tetrahydro-naphthalenyl group which might be unsubstituted, or substituted in the saturated ring with (C₁₋₄)alkyl, hydroxy, or phenyl, or substituted in the aromatic ring with one, two or three substituents independently selected from (C₁₋₄)alkyl, (C₁₋₄)alkoxy or halogen. Examples of 1,2,3,4-tetrahydro-naphthalenyl groups are 1,2,3,4-tetrahydro-naphthalen-1-yl, 2-methyl-1,2,3,4-tetrahydro-naphthalen-1-yl, 4-methyl-1,2,3,4-tetrahydro-naphthalen-1-yl and 5,7-dimethyl-1,2,3,4-tetrahydro-naphthalen-1-yl. Preferred are 1,2,3,4-tetrahydro-naphthalen-1-yl and 2-methyl-1,2,3,4-tetrahydro-naphthalen-1-yl.

The term “6,7,8,9-tetrahydro-5H-benzocycloheptenyl” means a 6,7,8,9-tetrahydro-5H-benzocycloheptenyl group which might be unsubstituted, or substituted in the saturated ring with (C₁₋₄)alkyl, hydroxy, or phenyl, or substituted in the aromatic ring with one, two or three substituents independently selected from (C₁₋₁₄)alkyl, (C₁₋₁₄)alkoxy or halogen. A preferred example of a 6,7,8,9-tetrahydro-5H-benzocycloheptenyl group is 6,7,8,9-tetrahydro-5H-benzocyclohepten-1-yl.

The term carboxy, alone or in combination, means a —COOH group.

The term “R⁸CO—” means for example CH₃(CO)—

The term “NR⁹R¹¹CO—” means for example NH₂CO—.

The term “NR⁹R¹¹COO—” means for example NH₂COO—, NH(CH₃)COO— and N(CH₃)₂COO—.

The term “R⁹R¹⁰N—” means for example NH₂—.

The term “R⁸OOC—” means for example CH₃OOC.

The term “R⁸SO₂NH—.” means for example CH₃SO₂NH—.

The term “R¹¹—CO—NH-” means for example CH₃CONH—.

The term “R⁸O—” means for example CH₃O—.

A further embodiment of the invention relates to the use of compounds of the general formula (I) as defined above, wherein the compounds are selected from:
2-{6,7-Dimethoxy-1-[2-(4-trifluoromethyl-phenyl)-ethyl]-3,4-dihydro-1H-isoquinolin-2-yl}-N-methyl-2-phenyl-acetamide;
(R)-2-{(S)-6,7-Dimethoxy-1-[2-(4-trifluoromethyl-phenyl)-ethyl]-3,4-dihydro-1H-isoquinolin-2-yl}-N-methyl-2-phenyl-acetamide;
2-[1-(3,4-Dimethoxy-benzyl)-5,8-dimethoxy-3,4-dihydro-1H-isoquinolin-2-yl]-N-(pyridin-2-yl-methyl)-acetamide;
2-[1-(3,4-dimethoxy-benzyl)-8-(cyclopropyl-methoxy)-5-methoxy-3,4-dihydro-1H-isoquinolin-2-yl]-N-(pyridin-2-yl-methyl)-acetamide;
2-[1-(3,4-dimethoxy-benzyl)-8-(2-fluoro-ethoxy)-5-methoxy-3,4-dihydro-1H-isoquinolin-2-yl]-N-(pyridin-2-yl-methyl)-acetamide;
2-[1-(3,4-dimethoxy-benzyl)-8-(2,2-difluoro-ethoxy)-5-methoxy-3,4-dihydro-1H-isoquinolin-2-yl]-N-(pyridin-2-yl-methyl)-acetamide;
2-[1-(3,4-dimethoxy-benzyl)-8-ethoxy-5-methoxy-3,4-dihydro-1H-isoquinolin-2-yl]-N-(pyridin-2-yl-methyl)-acetamide;
2-[1-(3,4-dimethoxy-benzyl)-8-propoxy-5-methoxy-3,4-dihydro-1H-isoquinolin-2-yl]-N-(pyridin-2-yl-methyl)-acetamide;
2-[1-(3,4-dimethoxy-benzyl)-8-allyloxy-5-methoxy-3,4-dihydro-1H-isoquinolin-2-yl]-N-(pyridin-2-yl-methyl)-acetamide;
2-[1-(3,4-dimethoxy-benzyl)-8-isopropoxy-5-methoxy-3,4-dihydro-1H-isoquinolin-2-yl]-N-(pyridin-2-yl-methyl)-acetamide; and
2-[1-(3,4-dimethoxy-benzyl)-5-propoxy-8-methoxy-3,4-dihydro-1H-isoquinolin-2-yl]-N-(pyridin-2-yl-methyl)-acetamide.

A further embodiment of the invention relates to the use of compounds of the general formula (I) as defined above, wherein the compounds are selected from:
2-[1-(3,4-Dimethoxy-benzyl)-6,7-dimethoxy-3,4-dihydro-1H-isoquinolin-2-yl]-N-benzyl-acetamide;
2-[1-(3,4-Dimethoxy-benzyl)-6,7-dimethoxy-3,4-dihydro-1H-isoquinolin-2-yl]-N-naphthalen-1-ylmethyl-acetamide;
2-[1-(3,4-Dimethoxy-benzyl)-6,7-dimethoxy-3,4-dihydro-1H-isoquinolin-2-yl]-N-(2-methoxy-benzyl)-acetamide;
2-[1-(3,4-Dimethoxy-benzyl)-6,7-dimethoxy-3,4-dihydro-1H-isoquinolin-2-yl]-N-(4-fluoro-benzyl)-acetamide;
2-[1-(3,4-Dimethoxy-benzyl)-6,7-dimethoxy-3,4-dihydro-1H-isoquinolin-2-yl]-N-(6-methoxy-naphthalen-2-ylmethyl)-acetamide;
2-[1-(3,4-Dimethoxy-benzyl)-6,7-dimethoxy-3,4-dihydro-1H-isoquinolin-2-yl]-N-(4-methoxy-naphthalen-2-ylmethyl)-acetamide;
2-[1-(3,4-Dimethoxy-benzyl)-6,7-dimethoxy-3,4-dihydro-1H-isoquinolin-2-yl]-N-(3,6)-difluoro-benzyl)-acetamide;
2-[1-(3,4-Dimethoxy-benzyl)-6,7-dimethoxy-3,4-dihydro-1H-isoquinolin-2-yl]-N-(1-phenyl-ethyl)-acetamide;
2-[1-(3,4-Dimethoxy-benzyl)-6,7-dimethoxy-3,4-dihydro-1H-isoquinolin-2-yl]-N-(pyridin-3-ylmethyl)-acetamide;
2-[1-(3,4-Dimethoxy-benzyl)-6,7-dimethoxy-3,4-dihydro-1H-isoquinolin-2-yl]-N-(2-methyl-benzyl)-acetamide;
2-[1-(3,4-Dimethoxy-benzyl)-6,7-dimethoxy-3,4-dihydro-1H-isoquinolin-2-yl]-N-(3-methyl-benzyl)-acetamide;
2-[1-(3,4-Dimethoxy-benzyl)-6,7-dimethoxy-3,4-dihydro-1H-isoquinolin-2-yl]-N-(indan-1-yl)-acetamide;
2-[1-(3,4-Dimethoxy-benzyl)-6,7-dimethoxy-3,4-dihydro-1H-isoquinolin-2-yl]-N-(1,2,3,4-tetrahydro-naphthalen-1-yl)-acetamide;
2-[1-(3,4-dimethoxy-benzyl)-6-methoxy-7-(pyrazin-2-yloxy)-3,4-dihydro-1H-isoquinolin-2-yl]-N-(indan-1-yl)-acetamide;
2-[1-(3,4-dimethoxy-benzyl)-6-methoxy-7-(thiazol-2-yloxy)-3,4-dihydro-1H-isoquinolin-2-yl]-N-(indan-1-yl)-acetamide;
2-[1-(3,4-Dimethoxy-benzyl)-6-methoxy-7-isopropoxy-3,4-dihydro-1H-isoquinolin-2-yl]-N-(5-methoxy-indan-1-yl)-acetamide;
2-[1-(3,4-Dimethoxy-benzyl)-6-methoxy-7-isopropoxy-3,4-dihydro-1H-isoquinolin-2-yl]-N-(6-methoxy-indan-1-yl)-acetamide;
2-[1-(3,4-Dimethoxy-benzyl)-6-methoxy-7-isopropoxy-3,4-dihydro-1H-isoquinolin-2-yl]-N-(6-methyl-indan-1-yl)-acetamide;
2-[1-(3,4-Dimethoxy-benzyl)-6-methoxy-7-isopropoxy-3,4-dihydro-1H-isoquinolin-2-yl]-N-(2-methyl-1,2,3,4-tetrahydronaphthalen-1-yl)-acetamide;
2-[1-(3,4-Dimethoxy-benzyl)-6-methoxy-7-isopropoxy-3,4-dihydro-1H-isoquinolin-2-yl]-N-(4-methyl-indan-1-yl)-acetamide;
2-[1-(3,4-Dimethoxy-benzyl)-6,7-dimethoxy-3,4-dihydro-1H-isoquinolin-2-yl]-N-(6-methoxy-indan-1-yl)-acetamide;
2-[1-(3,4-Dimethoxy-benzyl)-6,7-dimethoxy-3,4-dihydro-1H-isoquinolin-2-yl]-N-(6-methyl-indan-1-yl)-acetamide;
2-{1-[4-(pyrimidin-2-yloxy)-3-methoxy-benzyl]-6,7-dimethoxy-3,4-dihydro-1H-isoquinolin-2-yl}-N-benzyl-acetamide;
2-[1-(3,4-dimethoxy-benzyl)-6-methoxy-7-(N,N-dimethylcarbamoyloxy)-3,4-dihydro-1H-isoquinolin-2-yl]-N-(indan-1-yl)-acetamide;
2-[1-(3,4-dimethoxy-benzyl)-7-(3-fluoro-propoxy)-6-methoxy-3,4-dihydro-1H-isoquinolin-2-yl]-N-(indan-1-yl)-acetamide;
2-[1-(3,4-dimethoxy-benzyl)-7-(2-fluoro-ethoxy)-6-methoxy-3,4-dihydro-1H-isoquinolin-2-yl]-N-(indan-1-yl)-acetamide;
2-[1-(3,4-dimethoxy-benzyl)-7-(2,2-difluoro-ethoxy)-6-methoxy-3,4-dihydro-1H-isoquinolin-2-yl]-N-(indan-1-yl)-acetamide;
2-[1-(3,4-dimethoxy-benzyl)-7-(but-2-oxy)-6-methoxy-3,4-dihydro-1H-isoquinolin-2-yl]-N-(indan-1-yl)-acetamide;
2-[1-(3,4-dimethoxy-benzyl)-7-(cyclopropyl-methoxy)-6-methoxy-3,4-dihydro-1H-isoquinolin-2-yl]-N-(indan-1-yl)-acetamide;
2-[1-(3,4-dimethoxy-benzyl)-7-ethoxy-6-methoxy-3,4-dihydro-1H-isoquinolin-2-yl]-N-(indan-1-yl)-acetamide;
2-[1-(3,4-dimethoxy-benzyl)-7-propoxy-6-methoxy-3,4-dihydro-1H-isoquinolin-2-yl]-N-(indan-1-yl)-acetamide;
2-[1-(3,4-dimethoxy-benzyl)-7-allyloxy-6-methoxy-3,4-dihydro-1H-isoquinolin-2-yl]-N-(indan-1-yl)-acetamide;
2-[1-(3,4-dimethoxy-benzyl)-7-isopropoxy-6-methoxy-3,4-dihydro-1H-isoquinolin-2-yl]-N-(indan-1-yl)-acetamide;
2-[1-(3,4-dimethoxy-benzyl)-7-(1-methyl-prop-2-oxy)-6-methoxy-3,4-dihydro-1H-isoquinolin-2-yl]-N-benzyl-acetamide;
2-[1-(3,4-Dimethoxy-benzyl)-6-methoxy-7-isopropoxy-3,4-dihydro-1H-isoquinolin-2-yl]-N-[(1S)-indan-1-yl]-acetamide;
2-[1-(3,4-Dimethoxy-benzyl)-6-methoxy-7-isopropoxy-3,4-dihydro-1H-isoquinolin-2-yl]-N-benzyl-acetamide;
2-[(1S)-1-(3,4-Dimethoxy-benzyl)-6,7-dimethoxy-3,4-dihydro-1H-isoquinolin-2-yl]-N-[(1S)-indan-1-yl]-acetamide;
2-[1-(3,4-dimethoxy-benzyl)-7-ethoxy-6-methoxy-3,4-dihydro-1H-isoquinolin-2-yl]-N-benzyl-acetamide;
2-[1-(3,4-dimethoxy-benzyl)-7-propoxy-6-methoxy-3,4-dihydro-1H-iso quinolin-2-yl]-N-benzyl-acetamide;
2-[1-(3,4-dimethoxy-benzyl)-7-allyloxy-6-methoxy-3,4-dihydro-1H-isoquinolin-2-yl]-N-benzyl-acetamide;
N-benzyl-2-[1-(3,4-Dimethyl-benzyl)-6,7-dimethoxy-3,4-dihydro-1H-isoquinolin-2-yl]-acetamide;
2-[1-(3,4-Dimethyl-benzyl)-6,7-dimethoxy-3,4-dihydro-1H-isoquinolin-2-yl]-N-[(1S)-indan-1-yl]-acetamide;
N-benzyl-2-[1-(3,4-Diethyl-benzyl)-6,7-dimethoxy-3,4-dihydro-1H-isoquinolin-2-yl]-acetamide;
2-[1-(3,4-Diethyl-benzyl)-6,7-dimethoxy-3,4-dihydro-1H-isoquinolin-2-yl]-N-(pyridin-2-yl-methyl)-acetamide;
2-[1-(3,4-Diethyl-benzyl)-6,7-dimethoxy-3,4-dihydro-1H-isoquinolin-2-yl]-N-(pyridin-3-yl-methyl)-acetamide;
2-[1-(3,4-Diethyl-benzyl)-6,7-dimethoxy-3,4-dihydro-1H-isoquinolin-2-yl]-N-(pyridin-4-yl-methyl)-acetamide; and
2-[1-(3,4-Dichloro-benzyl)-6,7-dimethoxy-3,4-dihydro-1H-isoquinolin-2-yl]-N-(pyridin-3-yl-methyl)-acetamide.

A further embodiment of the invention relates to the use of compounds of the general formula (I) as defined above, wherein the compounds are selected from:
2-[1-(3,4-Dimethoxy-benzyl)-6,7-dimethoxy-3,4-dihydro-1H-isoquinolin-2-yl]-N-benzyl-acetamide;
2-[1-(3,4-Dimethoxy-benzyl)-6,7-dimethoxy-3,4-dihydro-1H-isoquinolin-2-yl]-N-naphthalen-1-ylmethyl-acetamide;
2-[1-(3,4-Dimethoxy-benzyl)-6,7-dimethoxy-3,4-dihydro-1H-isoquinolin-2-yl]-N-(indan-1-yl)-acetamide;
2-[1-(3,4-Dimethoxy-benzyl)-6,7-dimethoxy-3,4-dihydro-1H-isoquinolin-2-yl]-N-(1,2,3,4-tetrahydro-naphthalen-1-yl)-acetamide;
2-[1-(3,4-dimethoxy-benzyl)-6-methoxy-7-(pyrazin-2-yloxy)-3,4-dihydro-1H-isoquinolin-2-yl]-N-(indan-1-yl)-acetamide;
2-[1-(3,4-dimethoxy-benzyl)-6-methoxy-7-(thiazol-2-yloxy)-3,4-dihydro-1H-isoquinolin-2-yl]-N-(indan-1-yl)-acetamide;
2-[1-(3,4-Dimethoxy-benzyl)-6-methoxy-7-isopropoxy-3,4-dihydro-1H-isoquinolin-2-yl]-N-(5-methoxy-indan-1-yl)-acetamide;
2-[1-(3,4-Dimethoxy-benzyl)-6-methoxy-7-isopropoxy-3,4-dihydro-1H-isoquinolin-2-yl]-N-(6-methoxy-indan-1-yl)-acetamide;
2-[1-(3,4-Dimethoxy-benzyl)-6-methoxy-7-isopropoxy-3,4-dihydro-1H-isoquinolin-2-yl]-N-(6-methyl-indan-1-yl)-acetamide;
2-[1-(3,4-Dimethoxy-benzyl)-6-methoxy-7-isopropoxy-3,4-dihydro-1H-isoquinolin-2-yl]-N-(2-methyl-1,2,3,4-tetrahydronaphthalen-1-yl)-acetamide;
2-[1-(3,4-Dimethoxy-benzyl)-6-methoxy-7-isopropoxy-3,4-dihydro-1H-isoquinolin-2-yl]-N-(4-methyl-indan-1-yl)-acetamide;
2-[1-(3,4-Dimethoxy-benzyl)-6,7-dimethoxy-3,4-dihydro-1H-isoquinolin-2-yl]-N-(6-methoxy-indan-1-yl)-acetamide;
2-[1-(3,4-Dimethoxy-benzyl)-6,7-dimethoxy-3,4-dihydro-1H-isoquinolin-2-yl]-N-(6-methyl-indan-1-yl)-acetamide;
2-{1-[4-(pyrimidin-2-yloxy)-3-methoxy-benzyl]-6,7-dimethoxy-3,4-dihydro-1H-isoquinolin-2-yl}-N-benzyl-acetamide;
2-[1-(3,4-dimethoxy-benzyl)-6-methoxy-7-(N,N-dimethylcarbamoyloxy)-3,4-dihydro-1H-isoquinolin-2-yl]-N-(indan-1-yl)-acetamide;
2-[1-(3,4-dimethoxy-benzyl)-7-(3-fluoro-propoxy)-6-methoxy-3,4-dihydro-1H-isoquinolin-2-yl]-N-(indan-1-yl)-acetamide;
2-[1-(3,4-dimethoxy-benzyl)-7-(2-fluoro-ethoxy)-6-methoxy-3,4-dihydro-1H-isoquinolin-2-yl]-N-(indan-1-yl)-acetamide;
2-[1-(3,4-dimethoxy-benzyl)-7-(2,2-difluoro-ethoxy)-6-methoxy-3,4-dihydro-1H-isoquinolin-2-yl]-N-(indan-1-yl)-acetamide;
2-[1-(3,4-dimethoxy-benzyl)-7-(but-2-oxy)-6-methoxy-3,4-dihydro-1H-isoquinolin-2-yl]-N-(indan-1-yl)-acetamide;
2-[1-(3,4-dimethoxy-benzyl)-7-(cyclopropyl-methoxy)-6-methoxy-3,4-dihydro-1H-isoquinolin-2-yl]-N-(indan-1-yl)-acetamide;
2-[1-(3,4-dimethoxy-benzyl)-7-ethoxy-6-methoxy-3,4-dihydro-1H-isoquinolin-2-yl]-N-(indan-1-yl)-acetamide;
2-[1-(3,4-dimethoxy-benzyl)-7-propoxy-6-methoxy-3,4-dihydro-1H-isoquinolin-2-yl]-N-(indan-1-yl)-acetamide;
2-[1-(3,4-dimethoxy-benzyl)-7-allyloxy-6-methoxy-3,4-dihydro-1H-isoquinolin-2-yl]-N-(indan-1-yl)-acetamide;
2-[1-(3,4-dimethoxy-benzyl)-7-isopropoxy-6-methoxy-3,4-dihydro-1H-isoquinolin-2-yl]-N-(indan-1-yl)-acetamide;
2-[1-(3,4-dimethoxy-benzyl)-7-(1-methyl-prop-2-oxy)-6-methoxy-3,4-dihydro-1H-isoquinolin-2-yl]-N-benzyl-acetamide;
2-[1-(3,4-Dimethoxy-benzyl)-6-methoxy-7-isopropoxy-3,4-dihydro-1H-isoquinolin-2-yl]-N-[(1S)-indan-1-yl]-acetamide;
2-[1-(3,4-Dimethoxy-benzyl)-6-methoxy-7-isopropoxy-3,4-dihydro-1H-isoquinolin-2-yl]-N-benzyl-acetamide;
2-[(1S)-1-(3,4-Dimethoxy-benzyl)-6,7-dimethoxy-3,4-dihydro-1H-isoquinolin-2-yl]-N-[(1S)-indan-1-yl]-acetamide;
2-[1-(3,4-dimethoxy-benzyl)-7-ethoxy-6-methoxy-3,4-dihydro-1H-isoquinolin-2-yl]-N-benzyl-acetamide;
2-[1-(3,4-dimethoxy-benzyl)-7-propoxy-6-methoxy-3,4-dihydro-1H-isoquinolin-2-yl]-N-benzyl-acetamide;
2-[1-(3,4-dimethoxy-benzyl)-7-allyloxy-6-methoxy-3,4-dihydro-1H-isoquinolin-2-yl]-N-benzyl-acetamide;
N-benzyl-2-[1-(3,4-Dimethyl-benzyl)-6,7-dimethoxy-3,4-dihydro-1H-isoquinolin-2-yl]-acetamide;
2-[1-(3,4-Dimethyl-benzyl)-6,7-dimethoxy-3,4-dihydro-1H-isoquinolin-2-yl]-N-[(1S)-indan-1-yl]-acetamide;
N-benzyl-2-[1-(3,4-Diethyl-benzyl)-6,7-dimethoxy-3,4-dihydro-1H-isoquinolin-2-yl]-acetamide;
2-[1-(3,4-Diethyl-benzyl)-6,7-dimethoxy-3,4-dihydro-1H-isoquinolin-2-yl]-N-(pyridin-2-yl-methyl)-acetamide;
2-[1-(3,4-Diethyl-benzyl)-6,7-dimethoxy-3,4-dihydro-1H-isoquinolin-2-yl]-N-(pyridin-3-yl-methyl)-acetamide;
2-[1-(3,4-Diethyl-benzyl)-6,7-dimethoxy-3,4-dihydro-1H-isoquinolin-2-yl]-N-(pyridin-4-yl-methyl)-acetamide; and
2-[1-(3,4-Dichloro-benzyl)-6,7-dimethoxy-3,4-dihydro-1H-isoquinolin-2-yl]-N-(pyridin-3-yl-methyl)-acetamide;

A further embodiment of the invention relates to the use of compounds of the general formula (I) as defined above, wherein the compounds are selected from:
2-{6,7-dimethoxy-1-[2-(4-trifluoromethyl-phenyl)-ethyl]-3,4-dihydro-1H-isoquinolin-2-yl}-N-methyl-2-phenyl-acetamide;
(2R)-2-{(1S)-6,7-dimethoxy-1-[2-(4-trifluoromethyl-phenyl)-ethyl]-3,4-dihydro-1H-isoquinolin-2-yl}-N-methyl-2-phenyl-acetamide;
2-{6,7-dimethoxy-1-[2-(6-trifluoromethyl-pyridin-3-yl)-ethyl]-3,4-dihydro-1H-isoquinolin-2-yl}-N-methyl-2-phenyl-acetamide;
(2R)-2-{(1S)-6,7-dimethoxy-1-[2-(6-trifluoromethyl-pyridin-3-yl)-ethyl]-3,4-dihydro-1H-isoquinolin-2-yl}-N-methyl-2-phenyl-acetamide;
2-{1-[2-(3,4-Difluoro-phenyl)-ethyl]-6,7-dimethoxy-3,4-dihydro-1H-isoquinolin-2-yl}-2-phenyl-acetamide;
2-{1-[2-(2,3-Difluoro-phenyl)-ethyl]-6,7-dimethoxy-3,4-dihydro-1H-isoquinolin-2-yl}-2-phenyl-acetamide;
2-{6,7-Dimethoxy-1-[2-(4-trifluoromethyl-phenyl)-ethyl]-3,4-dihydro-1H-isoquinolin-2-yl}-2-phenyl-acetamide;
2-{6,7-Dimethoxy-1-[2-(3-methoxy-phenyl)-ethyl]-3,4-dihydro-1H-isoquinolin-2-yl}-2-phenyl-acetamide;
2-{1-[2-(2,5-Difluoro-phenyl)-ethyl]-6,7-dimethoxy-3,4-dihydro-1H-isoquinolin-2-yl}-2-phenyl-acetamide;
2-{6,7-Dimethoxy-1-[2-(2-methoxy-phenyl)-ethyl]-3,4-dihydro-1H-isoquinolin-2-yl}-2-phenyl-acetamide;
2-{1-[2-(4-Fluoro-phenyl)-ethyl]-6,7-dimethoxy-3,4-dihydro-1H-isoquinolin-2-yl}-2-phenyl-acetamide;
2-{1-[2-(3,4-Dimethoxy-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(2-methyl-5-trifluoromethyl-phenyl)-ethyl]-3,4-dihydro-1H-isoquinolin-2-yl}-2-phenyl-acetamide;
(R)-2-{(S)-1-[2-(3-Chloro-2-fluoro-phenyl)-ethyl]-6,7-dimethoxy-3,4-dihydro-1H-isoquinolin-2-yl}-2-phenyl-acetamide;
(R)-2-{(S)-1-[2-(3-Fluoro-4-methyl-phenyl)-ethyl]-6,7-dimethoxy-3,4-dihydro-1H-isoquinolin-2-yl}-2-phenyl-acetamide;
(R)-2-{(S)-1-[2-(4-Fluoro-2-trifluoromethyl-phenyl)-ethyl]-6,7-dimethoxy-3,4-dihydro-1H-isoquinolin-2-yl}-2-phenyl-acetamide;
(R)-2-{(S)-1-[2-(5-Fluoro-2-trifluoromethyl-phenyl)-ethyl]-6,7-dimethoxy-3,4-dihydro-1H-isoquinolin-2-yl}-2-phenyl-acetamide;
(R)-2-{(S)-1-[2-(2-Fluoro-5-trifluoromethyl-phenyl)-ethyl]-6,7-dimethoxy-3,4-dihydro-1H-isoquinolin-2-yl}-2-phenyl-acetamide;
(R)-2-{(S)-1-[2-(3-Fluoro-4-trifluoromethyl-phenyl)-ethyl]-6,7-dimethoxy-3,4-dihydro-1H-isoquinolin-2-yl}-2-phenyl-acetamide;
(R)-2-{(S)-1-[2-(4-Fluoro-3-methyl-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(2,3,5-trifluoro-phenyl)-ethyl]-3,4-dihydro-1H-isoquinolin-2-yl}-2-phenyl-acetamide;
(R)-2-{(S)-1-[2-(4-Fluoro-3-trifluoromethyl-phenyl)-ethyl]-6,7-dimethoxy-3,4-dihydro-1H-isoquinolin-2-yl}-2-phenyl-acetamide;
(R)-2-{(S)-1-[2-(3-Fluoro-5-trifluoromethyl-phenyl)-ethyl]-6,7-dimethoxy-3,4-dihydro-1H-isoquinolin-2-yl}-2-phenyl-acetamide;
(R)-2-{(S)-1-[2-(5-Chloro-2-trifluoromethyl-phenyl)-ethyl]-6,7-dimethoxy-3,4-dihydro-1H-isoquinolin-2-yl}-2-phenyl-acetamide;
(R)-2-{(S)-1-[2-(2-Fluoro-3-trifluoromethyl-phenyl)-ethyl]-6,7-dimethoxy-3,4-dihydro-1H-isoquinolin-2-yl}-2-phenyl-acetamide;
(R)-2-{(S)-1-[2-(2-Fluoro-4-trifluoromethyl-phenyl)-ethyl]-6,7-dimethoxy-3,4-dihydro-1H-isoquinolin-2-yl}-2-phenyl-acetamide;
(R)-2-{(S)-1-[2-(2-Difluoromethoxy-phenyl)-ethyl]-6,7-dimethoxy-3,4-dihydro-1H-isoquinolin-2-yl}-2-phenyl-acetamide;
(R)-2-{(S)-1-[2-(3-Fluoro-2-trifluoromethyl-phenyl)-ethyl]-6,7-dimethoxy-3,4-dihydro-1H-isoquinolin-2-yl}-2-phenyl-acetamide;
(R)-2-{(S)-1-[2-(2-Chloro-3-trifluoromethyl-phenyl)-ethyl]-6,7-dimethoxy-3,4-dihydro-1H-isoquinolin-2-yl}-2-phenyl-acetamide;
(R)-2-{(S)-1-[2-(2-Fluoro-6-trifluoromethyl-phenyl)-ethyl]-6,7-dimethoxy-3,4-dihydro-1H-isoquinolin-2-yl}-2-phenyl-acetamide;
(R)-2-{(S)-1-[2-(4-Chloro-3-trifluoromethyl-phenyl)-ethyl]-6,7-dimethoxy-3,4-dihydro-1H-isoquinolin-2-yl}-2-phenyl-acetamide;
(R)-2-{(S)-1-[2-(2,3-Difluoro-4-methyl-phenyl)-ethyl]-6,7-dimethoxy-3,4-dihydro-1H-isoquinolin-2-yl}-2-phenyl-acetamide;
(R)-2-{(S)-1-[2-(4-Difluoromethoxy-phenyl)-ethyl]-6,7-dimethoxy-3,4-dihydro-1H-isoquinolin-2-yl}-2-phenyl-acetamide;
(R)-2-{(S)-1-[2-(3,4-Dimethyl-phenyl)-ethyl]-6,7-dimethoxy-3,4-dihydro-1H-isoquinolin-2-yl}-2-phenyl-acetamide;
(R)-2-{(S)-1-[2-(3-Chloro-4-methyl-phenyl)-ethyl]-6,7-dimethoxy-3,4-dihydro-1H-isoquinolin-2-yl}-2-phenyl-acetamide;
(R)-2-{(S)-1-[2-(2-Chloro-6-fluoro-phenyl)-ethyl]-6,7-dimethoxy-3,4-dihydro-1H-isoquinolin-2-yl}-2-phenyl-acetamide;
(R)-2-[(S)-6,7-Dimethoxy-1-(2-o-tolyl-ethyl)-3,4-dihydro-1H-isoquinolin-2-yl]-2-phenyl-acetamide;
(R)-2-[(S)-6,7-Dimethoxy-1-(2r-m-tolyl-ethyl)-3,4-dihydro-1H-isoquinolin-2-yl]-2-phenyl-acetamide;
(R)-2-[(S)-6,7-Dimethoxy-1-(2-p-tolyl-ethyl)-3,4-dihydro-1H-isoquinolin-2-yl]-2-phenyl-acetamide;
(R)-2-{(S)-1-[2-(2-Fluoro-phenyl)-ethyl]-6,7-dimethoxy-3,4-dihydro-1H-isoquinolin-2-yl}-2-phenyl-acetamide;
(R)-2-{(S)-1-[2-(3-Fluoro-phenyl)-ethyl]-6,7-dimethoxy-3,4-dihydro-1H-isoquinolin-2-yl}-2-phenyl-acetamide;
(R)-2-{(S)-1-[2-(2,6-Dichloro-phenyl)-ethyl]-6,7-dimethoxy-3,4-dihydro-1H-isoquinolin-2-yl}-2-phenyl-acetamide;
(R)-2-{(S)-1-[2-(3,4-Dichloro-phenyl)-ethyl]-6,7-dimethoxy-3,4-dihydro-1H-isoquinolin-2-yl}-2-phenyl-acetamide;
(R)-2-{(S)-1-[2-(3,5-Dimethyl-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(2-trifluoromethyl-phenyl)-ethyl]-3,4-dihydro-1H-isoquinolin-2-yl}-2-phenyl-acetamide;
(R)-2-{(S)-1-[2-(2,4-Difluoro-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(2,3,6-trifluoro-phenyl)-ethyl]-3,4-dihydro-1H-isoquinolin-2-yl}-2-phenyl-acetamide;
(R)-2-{(S)-6,7-Dimethoxy-1-[2-(4-trifluoromethoxy-phenyl)-ethyl]-3,4-dihydro-1H-isoquinolin-2-yl}-2-phenyl-acetamide;
(R)-2-{(S)-1-[2-(2-Fluoro-3-methyl-phenyl)-ethyl]-6,7-dimethoxy-3,4-dihydro-1H-isoquinolin-2-yl}-2-phenyl-acetamide;
(R)-2-{(S)-1-[2-(4-Chloro-phenyl)-ethyl]-6,7-dimethoxy-3,4-dihydro-1H-isoquinolin-2-yl}-2-phenyl-acetamide;
(R)-2-{(S)-1-[2-(3-Chloro-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(3-trifluoromethoxy-phenyl)-ethyl]-3,4-dihydro-1H-isoquinolin-2-yl}-2-phenyl-acetamide;
(R)-2-{(S)-6,7-Dimethoxy-1-[2-(3,4,5-trifluoro-phenyl)-ethyl]-3,4-dihydro-1H-isoquinolin-2-yl}-2-phenyl-acetamide;
(R)-2-{(S)-6,7-Dimethoxy-1-[2-(3-trifluoromethyl-phenyl)-ethyl]-3,4-dihydro-1H-isoquinolin-2-yl}-2-phenyl-acetamide;
(R)-2-{(S)-6,7-Dimethoxy-1-[2-(2,3,4-trifluoro-phenyl)-ethyl]-3,4-dihydro-1H-isoquinolin-2-yl}-2-phenyl-acetamide;
(R)-2-{(S)-1-[2-(4-Bromo-2-fluoro-phenyl)-ethyl]-6,7-dimethoxy-3,4-dihydro-1H-isoquinolin-2-yl}-2-phenyl-acetamide;
(R)-2-{(S)-1-[2-(2,6-Difluoro-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(2-trifluoromethoxy-phenyl)-ethyl]-3,4-dihydro-1H-isoquinolin-2-yl}-2-phenyl-acetamide;
(R)-2-{(S)-1-[2-(2,4-Dichloro-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(2,4,5-trifluoro-phenyl)-ethyl]-3,4-dihydro-1H-isoquinolin-2-yl}-2-phenyl-acetamide;
(R)-2-{(S)-1-[2-(3-Bromo-phenyl)-ethyl]-6,7-dimethoxy-3,4-dihydro-1H-isoquinolin-2-yl}-2-phenyl-acetamide;
(R)-2-{(S)-1-[2-(4-tert-Butyl-phenyl)-ethyl]-6,7-dimethoxy-3,4-dihydro-1H-isoquinolin-2-yl}-2-phenyl-acetamide;
(R)-2-{(S)-1-[2-(2-Bromo-phenyl)-ethyl]-6,7-dimethoxy-3,4-dihydro-1H-isoquinolin-2-yl}-2-phenyl-acetamide;
(R)-2-{(S)-1-[2-(4-Bromo-phenyl)-ethyl]-6,7-dimethoxy-3,4-dihydro-1H-isoquinolin-2-yl}-2-phenyl-acetamide; and
(R)-2-{(S)-6,7-Dimethoxy-1-[2-(4-trifluoromethyl-phenyl)-ethyl]-3,4-dihydro-1H-isoquinolin-2-yl}-2-phenyl-acetamide.

A further preferred embodiment of the invention relates to the use of compounds of the general formula (I) as defined above, wherein the compounds are selected from:
2-{6,7-Dimethoxy-1-[2-(4-trifluoromethyl-phenyl)-ethyl]-3,4-dihydro-1H-isoquinolin-2-yl}-N-methyl-2-phenyl-acetamide, and
(R)-2-{(S)-6,7-Dimethoxy-1-[2-(4-trifluoromethyl-phenyl)-ethyl]-3,4-dihydro-1H-isoquinolin-2-yl}-N-methyl-2-phenyl-acetamide.

A further preferred embodiment of the invention relates to the use of compounds of the general formula (I) as defined above, wherein the compound is:
(R)-2-{(S)-6,7-Dimethoxy-1-[2-(4-trifluoromethyl-phenyl)-ethyl]-3,4-dihydro-1H-isoquinolin-2-yl}-N-methyl-2-phenyl-acetamide hydrochloride salt.

The present invention also includes the use of the above-mentioned compounds of general formula (I) and optically pure enantiomers, mixtures of enantiomers, racemates, optically pure diastereoisomers, mixtures of diastereoisomers, diastereoisomeric racemates, mixtures of diastereoisomeric racemates, or meso forms and pharmaceutically acceptable salts, solvent complexes and morphological forms thereof, for the preparation of a medicament to enhance and/or restore short-, middle- and/or long-term memory function and performance. Additionally, the above-mentioned compounds are also useful for the preparation of a medicament to enhance and/or restore short-, middle- and/or long-term memory function and performance. The present invention encompasses physiologically usable or pharmaceutically acceptable salts of compounds of general formula (I). This encompasses salts with physiologically compatible mineral acids such as hydrochloric acid, sulphuric or phosphoric acid; or with organic acids such as formic acid, methanesulphonic acid, acetic acid, trifluoroacetic acid, citric acid, fumaric acid, maleic acid, tartaric acid, succinic acid or salicylic acid and the like. The compounds of general formula (I) which are acidic (e.g. with a free carboxy group) can also form salts with physiologically compatible bases. Examples of such salts are alkali metal, alkali earth metal, ammonium and alkylammonium salts such as Na, K, Ca or tetraalkylammonium salt. The compounds of general formula (I) can also be present in the form of a zwitterion. For a comprehensive list see “Handbook of Pharmaceutical Salts”, P. H. Stahl, C. G. Wermuth Eds., Wiley-VCH, Weinheim/Zudrich 2002, p. 329-350.

The present invention encompasses different solvation complexes of compounds of general formula (I). The solvation can be effected in the course of the manufacturing process or can take place separately, e.g. as a consequence of hygroscopic properties of an initially anhydrous compound of general formula (I).

The present invention further encompasses different morphological forms, e.g. crystalline forms, of compounds of general formula (I) and their salts and solvation complexes. Particular heteromorphs may exhibit different dissolution properties, stability profiles, and the like, and are all included in the scope of the present invention.

The amount of the compound of the general formula (I) given to the patient to enhance and/or restore short-, middle- and/or long-term memory function and performance is comprised between 1 mg and 1000 mg per day (i.e. between 0.015 and 15 mg/kg body weight per day), particularly from 5 mg to 500 mg per day (i.e. 0.075 to 7.5 mg/kg per day), more particularly from 10 mg to 200 mg per day (i.e. 0.15 to 3 mg/kg per day).

The compounds of general formula (I) and their pharmaceutically usable salts can be used as medicament (e.g. in the form of pharmaceutical preparations). The pharmaceutical preparations can be administered internally, such as orally (e.g. in the form of tablets, coated tablets, dragees, hard and soft gelatine capsules, solutions, emulsions or suspensions), nasally (e.g. in the form of nasal sprays) or rectally (e.g. in the form of suppositories). However, the administration can also be effected parenterally, such as intramuscularly or intravenously (e.g. in the form of injection solutions).

The compounds of general formula (I) and their pharmaceutically usable salts can be processed with pharmaceutically inert, inorganic or organic adjuvants for the production of tablets, coated tablets, dragees, and hard gelatine capsules. Lactose, corn starch or derivatives thereof, talc, stearic acid or its salts etc. can be used, for example, as such adjuvants for tablets, dragees, and hard gelatine capsules.

Suitable adjuvants for soft gelatine capsules are, for example, vegetable oils, waxes, fats, semi-solid substances and liquid polyols, etc. Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils.

Suitable adjuvants for suppositories are, for example, natural or hardened oils, waxes, fats, semi-solid or liquid polyols.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The compounds obtained may also be converted into a pharmaceutically acceptable salt thereof in a manner known per se.

The compounds of general formula (I) may be useful for improving the occurrence of learning and/or memory deficits in a defect organism (e.g. as modelled in a lesioned mammal or aging mammal), and thus, altering or improving or restoring the learning ability and/or memory capacity of the organism. In a further embodiment, the compounds of the general formula (I) as described above may be used to prepare a medicament to enhance normal memory function (as in unlesioned, normal mammals used as animal models).

In a further embodiment, the compounds of the general formula (I) as described above may be used to prepare a medicament to treat patients who have been diagnosed as having or at risk of developing disorders in which diminished declarative memory is a symptom, e.g., as opposed to procedural memory.

In a further embodiment, the compounds of the general formula (I) as described above may be used to prepare a medicament for normal individuals for whom improved memory is desired. Memory disorders, which can be treated according to the present invention, may have a number of origins: a functional mechanism or clinical comorbidity (e.g. anxiety, depression), physiological aging (e.g. age-associated memory impairment, mild cognitive impairment, etc.), drug-induced or idiopathic anatomical lesions (e.g. dementia) or idiopathic anatomical lesions (e.g. dementia). Indications for which such preparations may be useful include learning disabilities and memory impairment due to, e.g., toxicant exposure, brain injury, age, schizophrenia, epilepsy, mental retardation in children, Down's Syndrome and senile dementia, including Alzheimer's disease. It can be used to treat Anterior Communicating Artery Syndrome and other stroke syndromes.

In a further embodiment, the compounds of the general formula (I) as described above may be used to prepare a medicament to treat the above-mentioned diseases, and further to treat (or lessen the severity of) or as a prophylaxis against memory impairment as a consequence or related to ischemia or hypoxia, such as may be the consequence of reduced blood flow or blood volume (including heart bypass surgery or diseases involving reduced or impaired cardiac output) or exposure to low oxygen conditions. In a further embodiment, the compounds of the general formula (I) as described above may be used to prepare a medicament to treat any clinical manifestations of cognitive dysfunction, expressed as deficits in any form or stage of attention, learning or memory linked to psychiatric disorders (e.g. schizophrenia or depression), neurodegenerative disorders, (e.g. Alzheimer or Parkinson) or any normal or pathological aging processes.

### EXPERIMENTAL SECTION

I. Chemistry:

The synthesis of the tetrahydroisoquinoline derivatives of the general formula (I) is described in WO 01/68609, WO 2004/085403 and WO 2005/118548.

II. Biology:

Compounds of general formula (I) were tested in accordance with the following experimental method.

Generation of Animal Paradigms to Test Agents

There are a variety of tests for cognitive function, especially learning and memory testing, which can be carried out using normal or lesioned animals. Learning and/or memory tests include, for example, motor skill learning, inhibitory avoidance, contextual fear conditioning, visual delay non-match to sample, spatial delay non-match to sample, visual discrimination, Barnes circular maze, Morris water maze, radial arm maze tests.

An exemplary motor skill learning test embodiment is the rotating rod paradigm. In this model, acquisition and retention of a motor task is assessed in groups of rats using a rotating rod paradigm consisting of placing an animal on a rotating horizontal metal rod, which accelerates from 4 to 40 rpm in two minutes. The rotating rod is placed 15 cm above a platform containing trip plates that control a digital timer. Time spent on the rotating rod is measured in seconds until a maximal cut-off time of 60 sec. Animals need repeated training until they are able to follow the accelerating movement of the bar for up to one minute. Four trials are given per day for several days.

An exemplary passive avoidance test utilizes an apparatus that consists of a lit chamber that can be separated from a dark chamber by a sliding door. At training, the animal is placed in the lit chamber for some period of time, and the door is opened. The animal moves to the dark chamber after a short delay—the latency—that is recorded. Upon entry into the dark chamber, the door is shut closed and a foot shock is delivered. Retention of the experience is determined after various time intervals, e.g., 24 or 48 hours, by repeating the test and recording the latency. There are many variants of the passive avoidance procedures

An exemplary maze testing embodiment is the water maze working memory test. In general, the method utilizes an apparatus, which consists of a circular water tank. A clear plexiglass platform, supported by a movable stand rest on the bottom of the tank, is submerged just below the water surface. Normally, a swimming rat cannot perceive the location of the platform but it may recall it from a previous experience and training, unless it suffers from some memory impairment. The time taken to locate the platform is measured and referred to as the latency. During the experiment, all orientational cues such as ceiling lights, etc., remain unchanged. Longer latencies are generally observed with rats with some impairment to their memory.

Generation of Animal Models of Cognitive Deficits:

Brain-lesioned animals (rodents or primates) can be used to identify dosages of the subject compositions, which restore memory consolidation. The lesioned mammal can have a lesion of the formix or a related brain structure that disrupts memory consolidation (e.g., perirhinal cortex, amygdala, medial septal nucleus, locus coeruleus, hippocampus, mammillary bodies). Lesions in the mammal can be produced by mechanical or chemical disruption. A complete transection of the formix disrupts adrenergic, cholinergic and GABAergic function and electrical activity, and induces morphological reorganization in the hippocampal formation. In general, the formix transection utilized in the subject method will not disconnect the parahippocampal region from the neocortex. In those embodiments, the formix transection will not disrupt functions that can be carried out by the parahippocampal region independent of processing by the hippocampal formation, and hence would not be expected to produce the full-blown amnesia seen following more complete hippocampal system damage in some tests.

The compounds of general formula (I) are administered to the animal in order to assess their effects on memory formation and/or memory consolidation. An increase in learned behaviour, relative to the absence of the test agents, indicates that the administered compound enhances memory formation and consolidation.

In the methods of the present invention, retention of the learned behavior can be determined, for example, after at least about 12-24 hours, 14-22 hours, 16-20 hours and or 18-19 hours after completion of the learning phase to determine whether the agents promote memory consolidation. In a particular embodiment, retention of the learned behavior can be determined 24 hours after completion of the learning phase.

As used herein, a “control mammal” can be an untreated lesioned mammal (i.e., a brain-lesioned animal receiving no agents or not the same combinations to be assessed), a trained control mammal (i.e., a mammal that undergoes training to demonstrate a learned behaviour without any lesion) and/or an untrained control mammal (i.e., a mammal with or without a lesion, that receives no training to demonstrate a learned behaviour).

Experiment 1:

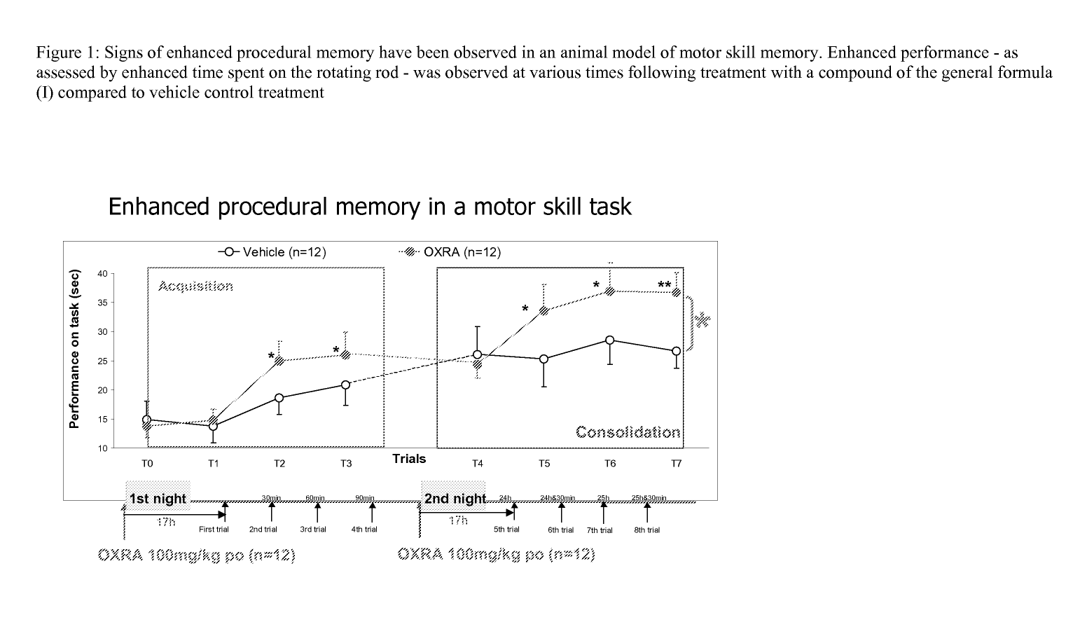

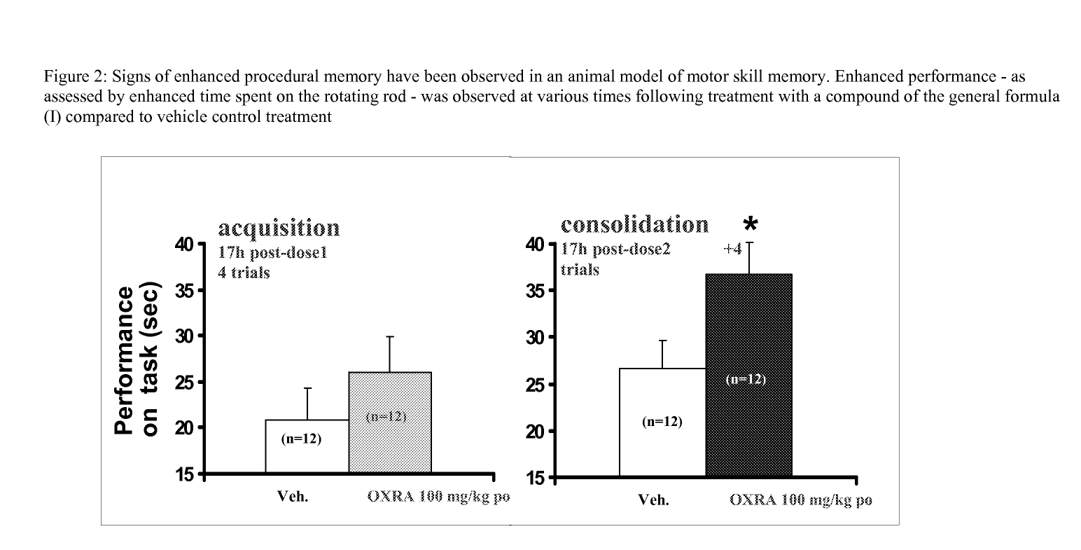

Signs of enhanced procedural memory have been observed in an animal model of motor skill memory. Enhanced performance—as assessed by enhanced time spent on the rotating rod—was observed at various times following treatment with a compound of the general formula (I) compared to vehicle control treatment (FIGS. 1 and 2; the abbreviation “po” means oral).

1. A method to enhance, maintain and/or restore all stages and/or types of short-, middle- and/or long-term memory comprising administering to a patient an effective amount of a compound of the general formula (I) in optically pure enantiomers, mixtures of enantiomers, racemates, optically pure diastereoisomers, mixtures of diastereoisomers, diastereoisomeric racemates, mixtures of diastereoisomeric racemates, meso forms, free or pharmaceutically acceptable salts, solvent complexes or morphological forms, 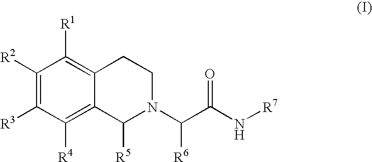
wherein
R¹ and R⁴ represent hydrogen;
R² and R³ independently represent (C₁₋₄)alkoxy;
R⁵ represents aryl-(C₁₋₄)alkyl or heteroaryl-(C₁₋₄)alkyl;
R⁶ represents a phenyl group;
R⁷ represents hydrogen or (C₁₋₄)alkyl.

2. The method according to claim 1 wherein
R¹ and R⁴ represent hydrogen;
R² and R³ represent methoxy;
R⁵ represents a 2-phenyl-ethyl- or a 2-pyridyl-ethyl group which groups are substituted with one or two substituents independently selected from methyl, trifluoromethyl or halogen;
R⁶ represents a phenyl group;
R⁷ represents hydrogen or (C₁₋₄)alkyl.

3. The method according to claim 1 wherein the compounds are selected from 2-{6,7-Dimethoxy-1-[2-(4-trifluoromethyl-phenyl)-ethyl]-3,4-dihydro-1H-isoquinolin-2-yl}-N-methyl-2-phenyl-acetamide, and (R)-2-{(S)-6,7-Dimethoxy-1-[2-(4-trifluoromethyl-phenyl)-ethyl]-3,4-dihydro-1H-isoquinolin-2-yl}-N-methyl-2-phenyl-acetamide.

4. The method according to claim 1 wherein said method enhances normal memory function.

5. The method according to claim 1 wherein said patient is a patients who has been diagnosed as having or being at risk of developing disorders in which diminished declarative memory is a symptom.

6. The method according to claim 1 wherein said method treats memory disorders.

7. The method according to claim 2 wherein said method enhances normal memory function.

8. The method according to claim 3 wherein said method enhances normal memory function.

9. The method according to claim 2 wherein said patient is a patient who has been diagnosed as having or being at risk of developing disorders in which diminished declarative memory is a symptom.

10. The method according to claim 3 wherein said patient is a patient who has been diagnosed as having or being at risk of developing disorders in which diminished declarative memory is a symptom.

11. The method according to claim 2 wherein said method treats memory disorders.

12. The method according to claim 3 wherein said method treats memory disorders.

13. The method according to claim 3 wherein said method enhances normal memory function.

14. The method according to claim 2, wherein the compounds are selected from 2-(6,7-Dimethoxy-1-[2-(4-trifluoromethyl-phenyl)-ethyl]-3,4-dihydro-1H-isoquinolin-2-yl)-N-methyl-2-phenyl-acetamide, and (R)-2-{(S)-6,7-Dimethoxy-1-[2-(4-trifluoromethyl-phenyl)-ethyl]-3,4-dihydro-1H-isoquinolin-2-yl}-N-methyl-2-phenyl-acetamide.
